(19) United States
(12) Patent Application Publication
(10) Pub. No.: US 2024/0378823 A1
(43) Pub. Date: Nov. 14, 2024

(54) SENSOR-INTEGRATED MEDICAMENT DEVICE FOR DATA TRANSFER INTO A VIRTUAL ENVIRONMENT

(71) Applicant: ResMed Digital Health Inc., San Diego, CA (US)

(72) Inventors: Nicholas James MEALEY, New York, NY (US); Audrey Eng BARTLETT, Allen, TX (US); Robert Louis BADDELEY, Madison, WI (US); Farah RAHMAN, San Diego, CA (US); William Baudrillart MASON, San Diego, CA (US); Sunjit KUMAR, Minneapolis, MN (US); Karl McCullough BROWN, Vienna, VA (US)

(21) Appl. No.: 18/658,854

(22) Filed: May 8, 2024

Related U.S. Application Data
(60) Provisional application No. 63/500,802, filed on May 8, 2023.

(51) Int. Cl. G06T 19/00; A61M 15/00; G01L 7/00; G02B 27/01; G16H 20/13

(52) U.S. Cl. CPC G06T 19/006; A61M 15/002; A61M 15/0021; G01L 7/00; G02B 27/017; G16H 20/13

(57) ABSTRACT

Various aspects of the subject technology related to systems, methods, and machine-readable media for utilizing an inhaler monitoring system is provided. The inhaler monitoring system may include a medicament device configured to provide medication to a user. The medicament device may include a mouthpiece, wherein the mouthpiece comprise a sensor configured to measure an airflow through the mouthpiece and generate a signal associated with the airflow. The sensor can be oriented parallel to the airflow through the mouthpiece. The system may include a processor and memory that stores instructions. The instructions can cause the processor to transmit the signal from the sensor, and facilitate an artificial reality environment. The system can include an artificial reality headset configured to permit the user to interact in the artificial reality environment.

### CROSS-RELATED APPLICATIONS

The present application claims the benefit of priority under 35 U.S.C. § 119 (e) from U.S. Provisional Patent Application Ser. No. 63/500,802 entitled “SENSOR-INTEGRATED MEDICAMENT DEVICE FOR DATA TRANSFER INTO A VIRTUAL ENVIRONMENT,” filed on May 8, 2023, the disclosure of which is hereby incorporated by reference in its entirety for all purposes.

### TECHNICAL FIELD

The present disclosure generally relates to a medicament device. More particularly, the present disclosure relates to a medicament device configured to transfer data to a virtual environment.

### BACKGROUND

Nebulizers and other medicament devices may be used to deliver various medications to the respiratory system of a patient to treat ailments from asthma, cystic fibrosis to chronic obstructive pulmonary disease (COPD). Such medicament devices are typically not connected to other medicament devices or the Internet.

In using these devices, adherence to a treatment plan may ensure effective use of the device. Many patients, however, do not know how to breathe correctly as part of their treatment plan using a medicament device. Patients may better adhere to their treatment plan by supervision under appropriate medical personnel.

### SUMMARY

One aspect of the present disclosure relates to a method for using an inhaler monitoring system, the system comprising a medicament device and an artificial reality headset. In an exemplary method, the method comprises receiving breathing data for a user recorded by a medicament device sensor, wherein the breathing data is converted to a transmission signal. The method includes determining a plurality of breathing parameters associated with the breathing data. The method includes generating an artificial reality environment associated with the plurality of breathing parameters, wherein the artificial reality environment is configured to display a user representation and provide accessibility to a shared artificial reality environment. The method can also include receiving the breathing data, generating an interactive protocol in the artificial reality environment.

According to another aspect of the present disclosure, an inhaler monitoring system is provided. The inhaler monitoring system may include a medicament device configured to provide medication to a user. The medicament device may include a mouthpiece, wherein the mouthpiece comprise a sensor configured to measure an airflow through the mouthpiece and generate a signal associated with the airflow. The sensor can be oriented parallel to the airflow through the mouthpiece. The system may include a processor and memory that stores instructions. The instructions can cause the processor to transmit the signal from the sensor, and facilitate an artificial reality environment. The system can include an artificial reality headset configured to permit the user to interact in the artificial reality environment.

According to yet other aspects of the present disclosure, a non-transitory computer-readable storage medium storing instructions encoded thereon that, when executed by a processor, cause the processor to perform operations, is provided. The operations may include receiving breathing data for a user recorded by a medicament device sensor, where in the breathing data is converted to a transmission signal. The operations may include determining a plurality of breathing parameters associated with the breathing data. The operations may include generating an artificial reality environment associated with the plurality of breathing parameters, wherein the artificial reality environment is configured to display a user representation and provide accessibility to a shared artificial reality environment. The operations may include receiving the breathing data, generating an interactive protocol in the artificial reality environment.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 shows an inhaler monitoring system for monitoring accurate, real-time medicament device usage, performing analytics on that data, and providing the data to other environments, according to one embodiment.

FIG. 2A is a schematic diagram of the inhaler monitoring system components with a head mounted display to facilitate an artificial reality environment.

FIG. 2B is a schematic diagram of the inhaler monitoring system components with an eyewear display to facilitate an augmented reality environment.

FIG. 3A is an isometric view of the mouthpiece of the medicament device. FIG. 3B is a sideview of the mouthpiece of the medicant device.

FIG. 4 is a high-level block diagram illustrating an example of a computing device used in either as a client device, application server, and/or database server, according to one embodiment.

FIG. 5 is an example flow diagram for implementing the medicament device in conjunction with artificial reality environment, according to certain aspects of the present disclosure.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regards to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the included clauses. Various aspects of the subject technology will now be disclosed according to particular but non-limiting examples. Various embodiments described in the present disclosure may be carried out in different ways and variations, and in accordance with a desired application or implementation.

In the following detailed description, numerous specific details are set forth to provide a full understanding of the present disclosure. It will be apparent, however, to one ordinarily skilled in the art, that embodiments of the present disclosure may be practiced without some of the specific details. In other instances, well-known structures and techniques have not been shown in detail so as not to obscure the disclosure.

In one aspect, unless otherwise stated, all measurements, values, ratings, positions, magnitudes, sizes, and other specifications that are set forth in this specification, including in the clauses that follow, are approximate, not exact. In one aspect, they are intended to have a reasonable range that is consistent with the functions to which they relate and with what is customary in the art to which they pertain. It is understood that some or all steps, operations, or processes may be performed automatically, without the intervention of a user. Method clauses may be provided to present elements of the various steps, operations, or processes in a sample order, and are not meant to be limited to the specific order or hierarchy presented.

The disclosed system aids a user of a medicament device in developing accuracy and adherence to a treatment plan. The system comprises a sensor that monitors the breathing of the user while using the medicament device. The data acquired from the medicament device may be used by a health care provider or other medical personnel to monitor and determine proper usage of the device. Further, based on the data, the health care provider may initiate protocols that adjust the treatment plan.

Another aspect of this disclosure allows patients to meet other patients where they may not be able to meet in person. For example, patients inflicted with cystic fibrosis are not allowed to meet in person due to cross-infection risks. Psychologically, such medical conditions may often result in depression and isolation. In other aspects the data acquired from the usage of the medicament device may be used in artificial reality environments, such as in augment reality (AR), virtual reality (VR), or other mixed reality (MR) environment. While in the artificial reality environment, the data from a user (patient) and/or community of users may be simulated.

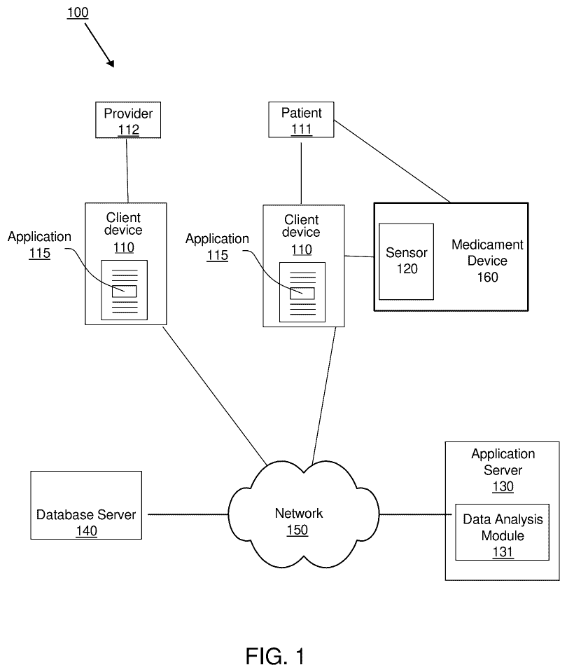

As depicted in FIG. 1, the system 100 may comprise an inhaler monitoring system 100. The inhaler monitoring system 100 includes one or more client devices 110, a medicament device sensor 120, a medicament device 160, an application server 130, database server 140, and a network 150. Although FIG. 1 illustrates only a single instance of most of the components of the inhaler monitoring system 100, in practice more than one of each component may be present, and additional or fewer components may be used.

The client devices 110, at the behest of their users, interact with the inhaler monitoring system 100 via the network 150. For purposes of explanation and clarity it is useful to identify at least two different types of users. A patient 111 is a user burdened with asthma who makes use of the inhaler monitoring system 100 at least in part to obtain personalized asthma rescue event risk notifications provided by the application server 130 and asthma management notifications created by their health care provider 112. Such notifications may be provided in exchange for the user's permission to allow the inhaler monitoring system 100 to monitor the patient's 111 medicament device 160 usage. As will be explained below, medication events are detected by a sensor 120 associated with the medicament device 160 and the user's client device 110, which in turn reports to the application server 130, which in turn may initiate a process to generate risk notifications which are provided to the user through the client device 110.

As mentioned earlier, a user may be a patient. Further, another type of user is a health care provider 112 who, with the patient's 111 express permission, also receives notifications regarding a patient's asthma management, as well as aggregated asthma community rescue event data and derived statistics regarding asthma events and other associated data. Other types of users are also contemplated, such as parents/guardians of patients 111 who may also want to receive notifications in the event that their own client devices 110 are distinct from that of their children.

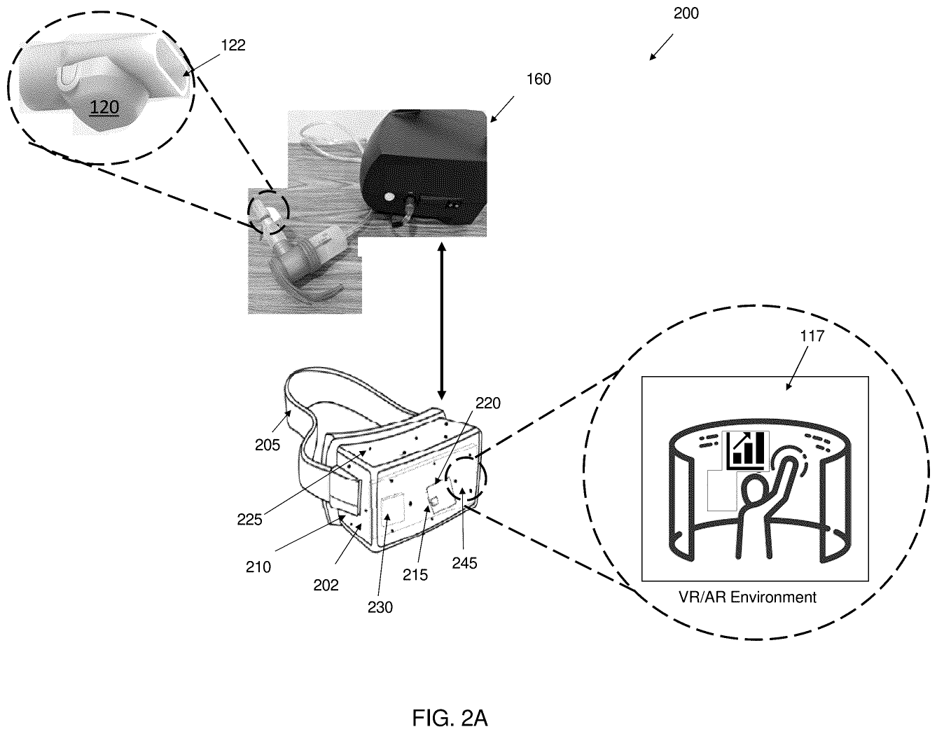

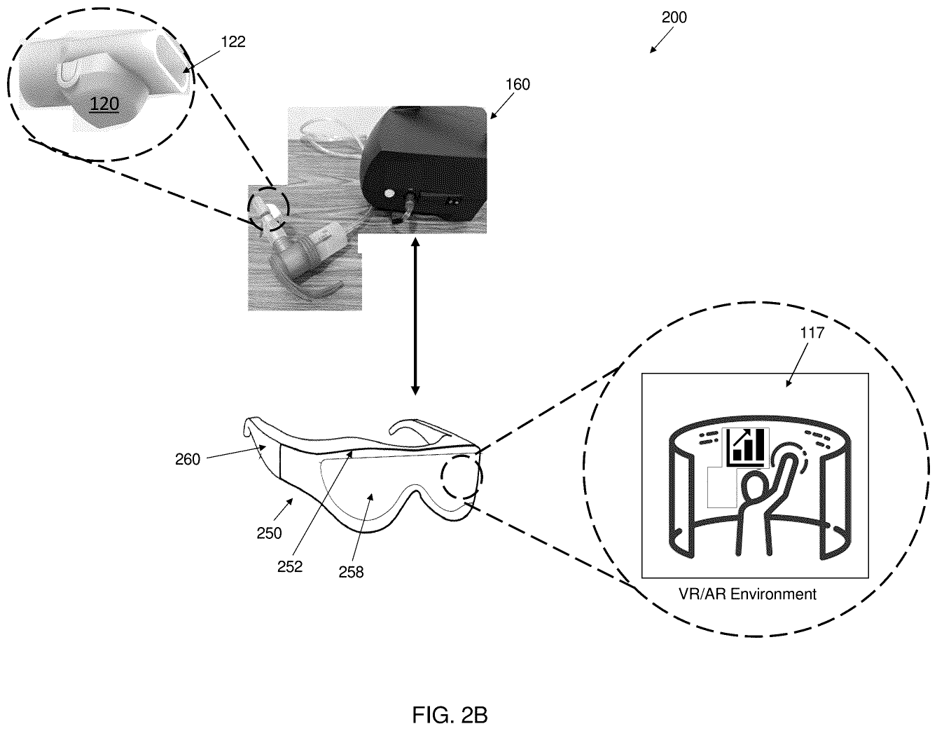

The client device 110 is a computer system. An example physical implementation is described more completely below with respect to FIG. 2. The client device 110 is configured to wirelessly communicate with the inhaler monitoring system 100 via network 150. With network 150 access, the client device 110 transmits to the system 100 the user's geographical location and the time of a rescue medication event, as well as information describing the event as received from the associated medicament device sensor 120 (referred to throughout as “sensor 120”).

Regarding user location and event times, the client device 110 may determine the geographical location and time of a rescue event through use of information about the cellular or wireless network 150 to which it is connected. For example, the current geographical location of the client device 110 may be determined by directly querying the software stack providing the network 150 connection. Alternatively, the geographical location information may be obtained by pinging an external web service (not shown in FIG. 1) made accessible via network 150. The time of an event may be provided by the sensor 120 as part of the event data or added to event data by querying an appropriate software routine available as part of the client device's 110 native operating system.

In addition to communicating with the application server 130, client devices 110 connected wirelessly to the inhaler monitoring system 100 may also exchange information with other connected client devices 110. For example, through a client software application 115, a health care provider 112 may receive a risk exacerbation notification describing a recent rescue event about a patient 111, then in response send a recommendation to the patient 111 for post-asthma rescue event treatment. Similarly, through application 115 patients 111 may communicate with their health care providers 112 and other patients 111.

Application 115 provides a user interface (herein referred to as a “dashboard”) that is displayed on a screen of the client device 110 and allows a user to input commands to control the operation of the application 115. The dashboard is the mechanism by which health care providers 112 and patients 111 access the system 100. For example, the dashboard allows patients 111 and providers 112 to interact with each other, receive asthma rescue event risk notifications in real-time, exchange messages about treatment, provide and receive additional event and non-event data, and so on. Application 115 may be coded as a web page, series of web pages, or content otherwise coded to render within an internet browser. Application 115 may also be coded as a proprietary application configured to operate on the native operating system of the client device 110.

Aspects of the present disclosure are directed to creating and administering artificial reality environments. For example, an artificial reality environment may be a shared artificial reality environment, a virtual reality (VR), an augmented reality environment, a mixed reality environment, a hybrid reality environment, a non immersive environment, a semi immersive environment, a fully immersive environment, and/or the like. The artificial environments may also include artificial collaborative working environments which include modes for interaction between various people or users in the artificial environments. The artificial environments of the present disclosure may provide elements that enable users to feel connected with other users. For example, audio and visual elements may be provided that maintain connections between various users that are engaged in the artificial environments. As used herein, “real-world” objects are non-computer generated and artificial or VR objects are computer generated. For example, a real-world space is a physical space occupying a location outside a computer and a real-world object is a physical object having physical properties outside a computer. For example, an artificial or VR object may be rendered and part of a computer generated artificial environment.

Embodiments of the disclosed technology may include or be implemented in conjunction with an artificial reality system. Artificial reality, extended reality, or extra reality (collectively “XR”) is a form of reality that has been adjusted in some manner before presentation to a user, which may include, e.g., virtual reality (VR), augmented reality (AR), mixed reality (MR), hybrid reality, or some combination and/or derivatives thereof. Artificial reality content may include completely generated content or generated content combined with captured content (e.g., real-world photographs). The artificial reality content may include video, audio, haptic feedback, or some combination thereof, any of which may be presented in a single channel or in multiple channels (such as stereo video that produces a three-dimensional effect to the viewer). Additionally, in some implementations, artificial reality may be associated with applications, products, accessories, services, or some combination thereof, that are, e.g., used to create content in an artificial reality and/or used in (e.g., perform activities in) an artificial reality. The artificial reality system that provides the artificial reality content may be implemented on various platforms, including a head-mounted display (HMD) connected to a host computer system, a standalone HMD, a mobile device or computing system, a “cave” environment or other projection system, or any other hardware platform capable of providing artificial reality content to one or more viewers.

“Virtual reality” or “VR,” as used herein, refers to an immersive experience where a user's visual input is controlled by a computing system. “Augmented reality” or “AR” refers to systems where a user views images of the real-world after they have passed through a computing system. For example, a tablet with a camera on the back can capture images of the real-world and then display the images on the screen on the opposite side of the tablet from the camera. The tablet can process and adjust or “augment” the images as they pass through the system, such as by adding virtual objects. AR also refers to systems where light entering a user's eye is partially generated by a computing system and partially composes light reflected off objects in the real-world. For example, an AR headset could be shaped as a pair of glasses with a pass-through display, which allows light from the real-world to pass through a waveguide that simultaneously emits light from a projector in the AR headset, allowing the AR headset to present virtual objects intermixed with the real objects the user can see. The AR headset may be a block-light headset with video pass-through. “Artificial reality,” “extra reality,” or “XR,” as used herein, refers to any of VR, AR, MR, or any combination or hybrid thereof.

In a further aspect, the application 115 may include instructions to generate the artificial reality environment. The artificial reality environment may be generated by the system processor of the medicament device. In yet another aspect, the application 115 configured to implement the artificial reality environment can be a component of the medicament device 160. Additional inputs for the artificial reality environment may be enhanced by the data received by the sensor 120. The sensor 120 may measure air flow data associated with the patient (user) breathing. The measurements from the airflow data may replicate the breathing conditions exhibited by the user as they traverse the artificial reality environment. For example, a high breathing rate detected by the sensor 120 may be replicated as the user running or exercising in the artificial reality environment 117. The parameters detected by the sensor 120 can also be used to develop an adherence protocol. The adherence protocol can be a tool used within the virtual reality environment 117 to aid the user in maintaining their treatment plan and provide notifications in the event that there is a divergence from a treatment plan. The user can interact with the adherence protocol in the artificial reality environment 117. For example, the artificial reality environment can contains a feature that allows the user to develop techniques to execute a Huff cough for maintaining an open air passage through the throat. In certain aspects, the artificial reality environment can comprise gaming aspects that can enhance the interaction between the user and the adherence protocol. In a further aspect, the artificial reality environment comprises a shared reality environment wherein the user can interact with others who have the ability to access the same artificial reality environment including healthcare providers and other patients.

In addition to providing the dashboard, application 115 may also perform some data processing on asthma rescue event data locally using the resources of client device 110 before sending the processed data through the network 150. Event data sent through the network 150 is received by the application server 130 where it is analyzed and processed for storage and retrieval in conjunction with database server 140. The application server 130 may direct retrieval and storage request to the database server 140 as required by the client application 115.

In another aspect, the client device 110 communicates with the sensor 120 using a network adapter and either a wired or wireless communication protocol, an example of which is the Bluetooth Low Energy (BTLE) protocol. BTLE is a short-ranged, low-powered, protocol standard that transmits data wirelessly over radio links in short range wireless networks. After the sensor 120 and client device 110 have been paired with each other using a BTLE passkey, the sensor 120 automatically synchronizes and communicate information relating to medicament device usage with the client device 110. If the sensor 120 has not been paired with a client device 110 prior to a rescue medication event, the information is stored locally until such a pairing occurs. Upon pairing, the sensor 120 communicates any stored event records to the client device 110. In other implementations, other types of wireless connections are used (e.g., infrared or 802.11).

Although client devices 110 and medicament devices 160 are described above as being separate physical devices (such as smart phones and inhalers, respectively), in the future it is contemplated that the medicament devices 160 may include not only sensors 120 integrated into a single housing with the device 160, but also aspects of the client device 110. For example, a medicament device 160 may include an audiovisual interface including a display or other lighting elements as well as speakers for presenting visual audible information. In such an implementation the medicament device 160 itself may present the contents of notifications provided by the application server 130 directly, in place of or in addition to presenting them through the client devices 110.

The medicament device 160 is a medical device used to deliver medication to the lungs of a user experiencing constricted respiratory airflow. Medicament devices (e.g., inhalers) are typically portable and small enough to be carried by hand for case of accessibility when treating respiratory attacks. In one embodiment, medicine is delivered in aerosol form through a medicament device 160 such as a metered dose inhaler. Metered dose inhalers included a pressured propellant canister of aerosol medicine, a metering valve for delivering a regulated medicine dosage amount, and a plastic holder that holds the pressurized canister and also forms a mouthpiece for delivery of the medicine. In another embodiment, medicine is delivered in dry powder form through a medicament device 160 such as a dry powder inhaler. Dry powder inhalers may have Cartesian ovular shaped bodies that house wheel and gear mechanisms enabling a user to index through a strip of dry powder medication. The bodies of dry powder inhalers also include a manifold and a mouthpiece 122 to deliver dry powder to the user. Examples of controller medications that are dispensed by a controller medicament device 160 include beclomethasone, budesonide, and fluticasone as well as combinations of those medications with a long-acting bronchodilator such as salmeterol or formoterol. Examples of rescue medications that are dispensed by a rescue medicament device 160 include albuterol, salbutamol, levalbuterol, metaproterenol, and terbutaline.

Each patient 111 may be associated with more than one medicament device 160. For example, the patient 111 may have a rescue medicament device 160 that dispenses rescue medication, and a controller medicament device 160 that dispenses controller medication. Similarly, each patient 111 may be associated with more than one sensor 120, each chosen to operate with one of the patient's medicament devices 160.

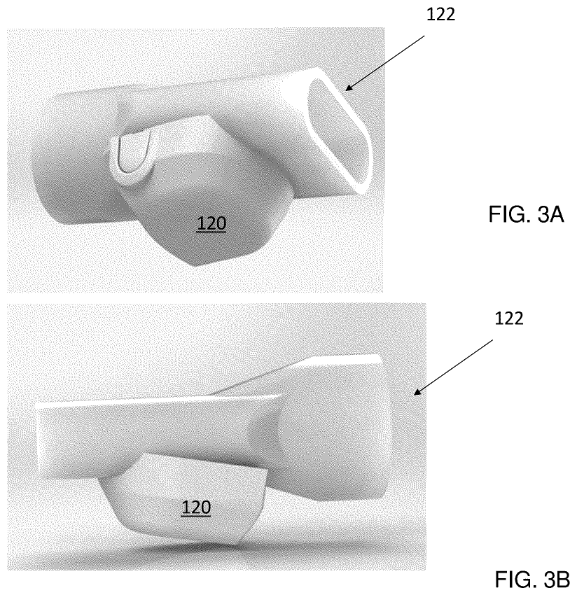

Generally, a sensor 120 is a physical device that monitors the usage of the medicament dispenser 160. The sensor 120 is either removably attachable to the medicament dispenser without impeding the operation of the medicament dispenser 160, or the sensor 120 is an integrated component that is a native part of the medicament dispenser 160 as made available by its manufacturer. The sensor 120 may also measure the airflow through the mouthpiece 122. As depicted in FIGS. 3A and 3B, the mouthpiece 122 can comprise a fixture that is structured and/or shaped to engage the patients mouth to inhale the medication. The sensor 120 may be coupled to the mouthpiece 122 such that the airflow is parallel to the orifices of the mouthpiece 122. In an aspect, the measurements by the sensor 120 may be taken while air flows through the orifices of the mouthpiece 122.

In another aspect, the sensor 120 includes its own network adapter (not shown) that communicates with the client device 110 either through a wired connection, or more typically through a wireless radio frequency connection. In one embodiment, the network adapter is a Bluetooth Low Energy (BTLE) wireless transmitter, however in other embodiments other types of wireless communication may be used (e.g., infrared, 802.11).

The sensor 120 may also be configured to communicate more directly with the application server 130. For example, if the network adapter of the sensor 120 is configured to communicate via a wireless standard such as 802.11 or LTE, the adapter may exchange data with a wireless access point such as a wireless router, which may in turn communicate with the application server 130 without necessarily involving the client device 110 in every exchange of data. These two methods of communicating are not mutually exclusive, and the sensor 120 may be configured to communicate with both the client device 110 and the application server 130. For example, in an embodiment of the system 100, the application server 130 may process an application with instructions to generate and run the VR, AR, or MR environment. In a further aspect, the system 100 may implement redundant transmission to ensure that event data arrives at the application server 130 or to provide information directly to the client device 110 while the application server 130 is determining what notification to provide in response to an event.

As introduced above, the sensor 120 captures data about usage of the medicament device 160 and airflow through the mouthpiece 122. Specifically, each sensor 120 captures the time and geographical location of the rescue medication event, that is, usages of the rescue medicament device 160, by the patient 111. Each sensor 120 transmits the event data in real-time or as soon as a network connection is achieved, automatically without input from the patient 111 or health care provider 112. The medication event information is sent to the application server 130 for use in analysis, generation of asthma rescue event notifications, and in aggregate analyses of event data across multiple patients. In a further aspect, the sensor can comprise a microphone to receive auditory data to assess the breathing characteristics of the user. For example, the microphone may be able to hear wheezing the user and correlate the wheezing to the rate and pattern of air flow detected by the sensor. In yet another aspect, the sensor may be able to determine the barometric pressure. For example, a correlation between the change in barometric measurement and other breathing data can be used to generate a responsive treatment by the medicament device 160, such as a change in medicine dosage or a notification sent to the user of the atmospheric conditions.

To accomplish this goal, there are a number of different ways for the sensor 120 to be constructed, and in part the construction will depend upon the construction of the medicament device 160 itself. Generally, all sensors 120 may include an onboard processor, persistent memory, and the network adapter mentioned above that together function to record, store, and report medication event information to the client device 110 and/or application server 130. Sensors 120 may also include a clock for recording the time and date of events.

Regarding specific sensor 120 constructions, traditional inhalers, such as mechanical dose counters, are not designed with sensors 120 in mind, and thus the sensor 120 may be constructed accordingly. Some implementations in this manner include mechanical, electrical, or optical sensors to detect movement of the medicament device 160, priming of the medicament device 160, activation of the medicament device 160, inhalation by the user, etc. In contrast, modern inhalers, such as deflectable membrane dose counters, include electrical circuitry may report event information as an electrical data signal which a sensor 120 is designed to receive and interpret, for example the medicament device 160 itself may report movement, priming, and activation to the sensor 120.

The application server 130 may be a computer or network of computers. Although a simplified example is illustrated in FIG. 2, typically the application server 130 will be a server class system that uses powerful processors, large memory, and faster network components compared to a typical computing system used, for example, as a client device 110. The application server 130 typically has large secondary storage, for example, using a RAID (redundant array of independent disks) array and/or by establishing a relationship with an independent content delivery network (CDN) contracted to store, exchange and transmit data such as the asthma notifications contemplated above. Additionally, the computing system includes an operating system, for example, a UNIX operating system, LINUX operating system, or a WINDOWS operating system. The operating system manages the hardware and software resources of the application server 130 and also provides various services, for example, process management, input/output of data, management of peripheral devices, and so on. The operating system provides various functions for managing files stored on a device, for example, creating a new file, moving or copying files, transferring files to a remote system, and so on.

The application server 130 includes a software architecture for supporting access and use of inhaler monitoring system 100 by many different client devices 110 through network 150, and thus at a high level may be generally characterized as a cloud-based system. The application server 130 generally provides a platform for patients 111 and health care providers 112 to report data recorded by the sensors 120 associated with their medicament devices 160 including both rescue medication and controller medication events, collaborate on asthma treatment plans, browse and obtain information relating to their condition and geographic location, and make use of a variety of other functions.

Generally, the application server 130 is designed to handle a wide variety of data. The application server 130 includes logical routines that perform a variety of functions including checking the validity of the incoming data, parsing and formatting the data if necessary, passing the processed data to a database server 140 for storage, and confirming that the database server 140 has been updated.

The application server 130 stores and manages data at least in part on a patient-by-patient basis. Towards this end, the application server 130 creates a patient profile for each user. The patient profile is a set of data that characterizes a patient 111 of the inhaler monitoring system 100. The patient profile may include identifying information about the patient such as age, gender, current rescue medication, current controller medication, notification preferences, a controller medication adherence plan, a patient's relevant medical history, and a list of non-patient users authorized to access to the patient profile. The profile may further specify a device identifier, such as a unique media access control (MAC) address identifying the one or more client devices 110 or sensors 120 authorized to submit data (such as controller and rescue medication events) for the patient.

The profile may specify which different types of notifications are provided to patients 111 and their personal health care providers 112, as well as the frequency with which notifications are provided. The notifications can also be based on whether a measured value for a breathing parameter (e.g. breathing frequency, tidal volume, flowrate, and pulmonary pressure) has exceeded or fell below a threshold. For example, the breathing flowrate measurement comparison to a threshold value can trigger a notification and/or an adjustment in the artificial reality environment.

A patient 111 may also authorize a health care provider 112 to receive notifications indicating a rescue event. The patient 111 may also authorize their health care provider 112 be given access to their patient profile and rescue event history. If the health care provider 112 is provided access to the patient profile of the patient 111, the health care provider may specify controller adherence or rescue medication plans. Medication plans may include a prescribed number of doses per day for controller medications.

The application server 130 also creates profiles for health care providers 112. A health care provider profile may include identifying information about the health care provider 112, such as the office location, qualifications and certifications, and so on. The health care provider profile also includes information about their patient population. The provider profile may include access to all of the profiles of that provider's patients, as well as derived data from those profiles such as aggregate demographic information, rescue and controller medication event patterns, and so on. This data may be further subdivided according to any type of data stored in the patient profiles, such as by geographic area (e.g., neighborhood, city) over a time period (e.g., weekly, monthly, yearly).

The application server 130 receives rescue medication event information from the client device 110 or the sensor 120, triggering a variety of routines on the application server 130. In the example implementations described below, the data analysis module 131 executes routines to access asthma event data as well as other data including a patient's profile, analyze the data, and output the results of its analysis to both patients 111 and providers 112. This process is generally referred to as an asthma risk analysis. The asthma risk analysis may be performed at any point in time, in response to a rescue event, due to a relevant change in the patient's environment, and in response to any one of a number of triggering conditions discussed further below.

Other analyses are also possible. For example, a risk analysis may be performed on rescue and controller medication use for multiple patients to identify based on spatial/temporal clusters (or outbreaks) of medication use based on historically significant permutations from individual, geographic, clinical, epidemiologic, demographic, or spatial or temporal baselines or predicted or expected values. Other types of analyses may include daily/weekly adherence trends, adherence changes over time, adherence comparisons to other relevant populations (e.g., all patients, patients on a particular rescue medication or controller medication or combination thereof, identification of triggers (spatial, temporal, environmental), rescue use trends over time, and rescue use comparisons to other relevant populations.

Responsive to any analyses performed, the application server 130 prepares and delivers push notifications to send to patients 111, authorized health care providers 112, and/or other users provided access to the patient's profile. Notifications may provide details about the timing, location, and affected patient(s) 111 involved in a medication rescue event. Notifications may additionally comprise a distress or emergency signal that requests emergency assistance that are distributed to emergency assistance providers 112. Notifications may also include the results of the asthma risk analysis performed by the data analysis module 131. More information regarding the types of notifications that may be sent and the content they may contain is further described below.

In addition to providing push notifications in response to an asthma risk analysis, notifications may also be provided as pull notifications, at particular time intervals. Additionally, some notifications (whether push or pull) may be triggered not in response to an asthma risk analysis performed in response to a rescue medication event, but instead in response to a risk analysis performed in response to one of the underlying factors in the asthma risk analysis changing, such that an updated notification is warranted. For example, if weather conditions indicate that an increase in air pollution is occurring or is imminent, this may trigger the carrying out of asthma risk analyses for all patients located in the particular geographic area where the pollution is occurring.

Notifications are provided through the network 150 to client applications 115 in a data format specifically designed for use with the client applications, and additionally or alternatively may be provided as short message service (SMS) messages, emails, phone calls, or in other data formats communicated using other communication mediums.

The database server 140 stores patient and provider data related data such as profiles, medication events, patient medical history (e.g., electronic medical records). Patient and provider data is encrypted for security and is at least password protected and otherwise secured to meet all Health Insurance Portability and Accountability Act (HIPAA) requirements. Any analyses (e.g., asthma risk analyses) that incorporate data from multiple patients (e.g., aggregate rescue medication event data) and are provided to users is de-identified so that personally identifying information is removed to protect patient privacy.

The database server 140 also stores non-patient data used in asthma risk analyses. This data includes regional data about a number of geographic regions such as public spaces in residential or commercial zones where patients are physically located and may be exposed to pollutants. This data may specifically include or be processed to obtain a patient's proximity to green space (areas including concentrated numbers of trees and plants). One example of regional data includes georeferenced weather data, such as temperature, wind patterns, humidity, the air quality index, and so on. Another example is georeferenced pollution data, including particulate counts for various pollutants at an instance of time or measured empirically. The regional data includes information about the current weather conditions for the time and place of the rescue event such as temperature, humidity, air quality index. All of the items of data above may vary over time, and as such the data itself may be indexed by time, for example separate data points may be available by time of day (including by minute or hour), or over longer periods such as by day, week, month, or season. Although the database server 140 is illustrated in FIG. 1 as being an entity separate from the application server 130 the database server 140 may alternatively be a hardware component that is part of another server such as application server 130, such that the database server 140 is implemented as one or more persistent storage devices, with the software application layer for interfacing with the stored data in the database is a part of that other application server 130.

The database server 140 stores data according to defined database schemas. Typically, data storage schemas across different data sources vary significantly even when storing the same type of data including cloud application event logs and log metrics, due to implementation differences in the underlying database structure. The database server 140 may also store different types of data such as structured data, unstructured data, or semi-structured data. Data in the database server 140 may be associated with users, groups of users, and/or entities. The database server 140 provides support for database queries in a query language (e.g., SQL for relational databases, JSON NoSQL databases, etc.) for specifying instructions to manage database objects represented by the database server 140, read information from the database server 140, or write to the database server 140.

The network 150 represents the various wired and wireless communication pathways between the client 110 devices, the sensor 120, the application server 130, and the database server 140. Network 150 uses standard Internet communications technologies and/or protocols. Thus, the network 150 may include links using technologies such as Ethernet, IEEE 802.11, integrated services digital network (ISDN), asynchronous transfer mode (ATM), etc. Similarly, the networking protocols used on the network 150 may include the transmission control protocol/Internet protocol (TCP/IP), the hypertext transport protocol (HTTP), the simple mail transfer protocol (SMTP), the file transfer protocol (FTP), etc. The data exchanged over the network 150 may be represented using technologies and/or formats including the hypertext markup language (HTML), the extensible markup language (XML), etc. In addition, all or some links may be encrypted using conventional encryption technologies such as the secure sockets layer (SSL), Secure HTTP (HTTPS) and/or virtual private networks (VPNs). In another embodiment, the entities may use custom and/or dedicated data communications technologies instead of, or in addition to, the ones described above.

The system 200 can also include an integration between the medicament device and an artificial reality headset, according to certain aspects of the present disclosure. FIG. 2A is a diagram of an artificial reality head-mounted display (HMD) 202. The HMD 202 includes a front rigid body 205 and a band 210. The front rigid body 205 includes one or more electronic display elements of an electronic display 245, an inertial motion unit (IMU) 215, one or more position sensors 220, locators 225, and one or more compute units 230. The position sensors 220, the IMU 215, and compute units 230 may be internal to the HMD 202 and may not be visible to the user. In various implementations, the IMU 215, position sensors 220, and locators 225 can track movement and location of the HMD 202 in the real-world and in a virtual environment in three degrees of freedom (3DoF), six degrees of freedom (6DoF), etc. For example, the locators 225 can emit infrared light beams which create light points on real objects around the HMD 202. As another example, the IMU 215 can include e.g., one or more accelerometers, gyroscopes, magnetometers, other non-camera-based position, force, or orientation sensors, or combinations thereof. One or more cameras (not shown) integrated with the HMD 202 can detect the light points. Compute units 230 in the HMD 202 can use the detected light points to extrapolate position and movement of the HMD 202 as well as to identify the shape and position of the real objects surrounding the HMD 202.

The electronic display 245 can be integrated with the front rigid body 205 and can provide image light to a user as dictated by the compute units 230. In various embodiments, the electronic display 245 can be a single electronic display or multiple electronic displays (e.g., a display for each user eye). Examples of the electronic display 245 include: a liquid crystal display (LCD), an organic light-emitting diode (OLED) display, an active-matrix organic light-emitting diode display (AMOLED), a display including one or more quantum dot light-emitting diode (QOLED) sub-pixels, a projector unit (e.g., microLED, LASER, etc.), some other display, or some combination thereof.

In some implementations, the HMD 202 can be coupled to a core processing component (not shown) to facilitate the artificial reality environment, wherein the core processing component is located in a client device 110 or external application server 130. In another aspect, the core processing unit used the facilitate the artificial reality environment can be a component of the medicament device 160. Further, the HMD can be coupled to and/or one or more external sensors (not shown). The external sensors can monitor the HMD 202 (e.g., via light emitted from the HMD 202) which the PC can use, in combination with output from the IMU 215 and position sensors 220, to determine the location and movement of the HMD 202.

FIG. 2B is a diagram of system which includes an augmented reality eyewear 250. The augmented reality eyewear 250 can a pass-through display 258 and a frame 260. The frame 260 can house various electronic components (not shown) such as light projectors (e.g., LASERs, LEDs, etc.), cameras, eye-tracking sensors, MEMS components, networking components, etc. The projectors can be coupled to the pass-through display 258, e.g., via optical elements, to display media to a user. The optical elements can include one or more waveguide assemblies, reflectors, lenses, mirrors, collimators, gratings, etc., for directing light from the projectors to a user's eye. Controllers in the HMD 252 can convert the image data into light pulses from the projectors, which can be transmitted via the optical elements as output light to the user's eye. The output light can mix with light that passes through the pass-through display 258, allowing the output light to present virtual objects that appear as if they exist in the real-world.

Similarly to the HMD 202 in FIG. 2A, the augmented reality eyewear 250 in FIG. 2B can also include motion and position tracking units, cameras, light sources, etc., which allow the augmented reality eyewear 250 to, e.g., track itself in 3DoF or 6DoF, track portions of the user (e.g., hands, feet, head, or other body parts), map virtual objects to appear as stationary as the frame 260 moves, and have virtual objects react to gestures and other real-world objects. For example, the mixed eyewear 250 can track relative position of various important features of the user or between users, such as eyes and hands. As an example, the augmented reality eyewear 250 may include a relational coordinate system to track the relative positions of the eyes and hands for one or more user representations and include personal coordinate systems for personalizing remaining portions of an artificial reality environment for each user. In this way, the augmented reality eyewear 250 can maintain (or purposefully avoid) eye contact between user representations across all coordinate systems. That is, the personal coordinate system may implement user specific perception preferences while maintaining the same relative positions of the various important features.

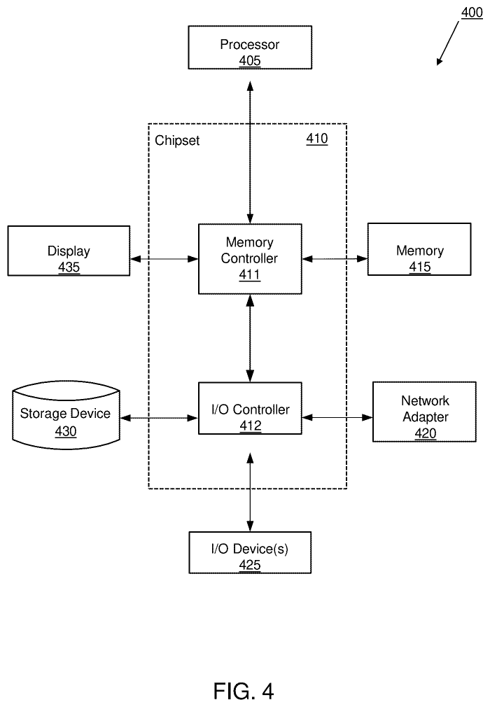

FIG. 4 is a high-level block diagram illustrating physical components of an example computer 400 that may be used as part of a client device 110, application server 130, medicament device 160 and/or database server 140 from FIG. 1, according to one embodiment. Illustrated is a chipset 410 coupled to at least one processor 405. Coupled to the chipset 410 is volatile memory 415, a network adapter 420, an input/output (I/O) device(s) 425, a storage device 430 representing a non-volatile memory, and a screen display 435. In one embodiment, the functionality of the chipset 410 is provided by a memory controller 411 and an I/O controller 412. In another embodiment, the memory 415 is coupled directly to the processor 405 instead of the chipset 410. In some embodiments, memory 415 includes high-speed random-access memory (RAM), such as DRAM, SRAM, DDR RAM or other random-access solid-state memory devices.

The storage device 430 is any non-transitory computer-readable storage medium, such as a hard drive, compact disk read-only memory (CD-ROM), DVD, or a solid-state memory device. The memory 415 holds instructions and data used by the processor 405. The I/O device 425 may be a touch input surface (capacitive or otherwise), a mouse, track ball, or other type of pointing device, a keyboard, or another form of input device. The screen display 435 displays images and other information from for the computer 400. The network adapter 420 couples the computer 400 to the network 150. As is known in the art, a computer 400 may have different and/or other components than those shown in FIG. 2. In addition, the computer 400 may lack certain illustrated components. In one embodiment, a computer 400 acting as database server 140 may lack a dedicated I/O device 425, and/or screen display 435. Moreover, the storage device 430 may be local and/or remote from the computer 400 (such as embodied within a storage area network (SAN)), and, in one embodiment, the storage device 430 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analyses introduced above. In one embodiment, the processing power of the application server 130 provided by a service such as Amazon Web Services™. Also in contrast, the database server 140 may include many, physically separate computers each having a significant amount of persistent storage capacity for storing the data associated with the application server.

As is known in the art, the computer 400 is adapted to execute computer program modules for providing functionality described herein. A module may be implemented in hardware, firmware, and/or software. In one embodiment, program modules are stored on the storage device 430, loaded into the memory 415, and executed by the processor 405.

The computer system 400 can include, in addition to hardware, code that creates an execution environment for the computer program in question, e.g., code that constitutes processor firmware, a protocol stack, a database management system, an operating system, or a combination of one or more of them stored in an included memory 415, such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device for storing information and instructions to be executed by processor 405. The processor 405 and the memory 415 can be supplemented by, or incorporated in, special purpose logic circuitry.

The instructions may be stored in the memory 415 and implemented in one or more computer program products, i.e., one or more modules of computer program instructions encoded on a computer-readable medium for execution by, or to control the operation of, the computer system 400, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 415 may also be used for storing temporary variable or other intermediate information during execution of instructions to be executed by the processor 405.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

The computer system 400 further includes a data storage device 430 such as a magnetic disk or optical disk. The computer system 400 may be coupled via input/output controller 412 to various devices. The input/output controller 412 can be any input/output device. Exemplary input/output modules 412 include data ports such as USB ports. The input/output controller 412 is configured to connect to a network adapter 420. Exemplary network adapter 220 include networking interface cards, such as Ethernet cards and modems. In certain aspects, the input/output controller 412 is configured to connect to a plurality of devices. Exemplary input devices 425 include a keyboard and a pointing device, e.g., a mouse or a trackball, by which a user can provide input to the computer system 400. Other kinds of input devices can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback, and input from the user can be received in any form, including acoustic, speech, tactile, or brain wave input. Exemplary output devices 425 include display devices such as an LCD (liquid crystal display) monitor, or augmented reality headset for displaying information to the user.

According to one aspect of the present disclosure, the above-described systems can be implemented using a computer system 400 in response to the processor 405 executing one or more sequences of one or more instructions contained in the memory 415. Such instructions may be read into memory 415 from another machine-readable medium, such as data storage device 430. Execution of the sequences of instructions contained in the main memory 415 causes the processor 405 to perform the process steps described herein. One or more processors in a multi-processing arrangement may also be employed to execute the sequences of instructions contained in the memory 415. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., such as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

The computer system 400 can include clients and servers. A client and server are generally remote from each other and typically interact through a communication network. The relationship of client and server arises by virtue of computer programs running on the respective computers and having a client-server relationship to each other. The computer system 400 can be, for example, and without limitation, a desktop computer, laptop computer, or tablet computer. The computer system 400 can also be embedded in another device, for example, and without limitation, a mobile telephone, a PDA, a mobile audio player, a Global Positioning System (GPS) receiver, a video game console, medicament device 160 and/or a television set top box.

The term “machine-readable storage medium” or “computer-readable medium” as used herein refers to any medium or media that participates in providing instructions to the processor 405 for execution. Such a medium may take many forms, including, but not limited to, non-volatile media, volatile media, and transmission media. Non-volatile media include, for example, optical or magnetic disks, such as the data storage device 430. Volatile media include dynamic memory, such as the memory 415. Transmission media include coaxial cables, copper wire, and fiber optics. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As the user computing system 400 reads XR data and provides an artificial reality, information may be read from the XR data and stored in a memory device, such as the memory 415. Additionally, data from the memory 415 servers accessed via a network, the network adapter 420, or the data storage 430 may be read and loaded into the memory 415. Although data is described as being found in the memory 415, it will be understood that data does not have to be stored in the memory 415 and may be stored in other memory accessible to the processor 405 or distributed among several media, such as the data storage 430.

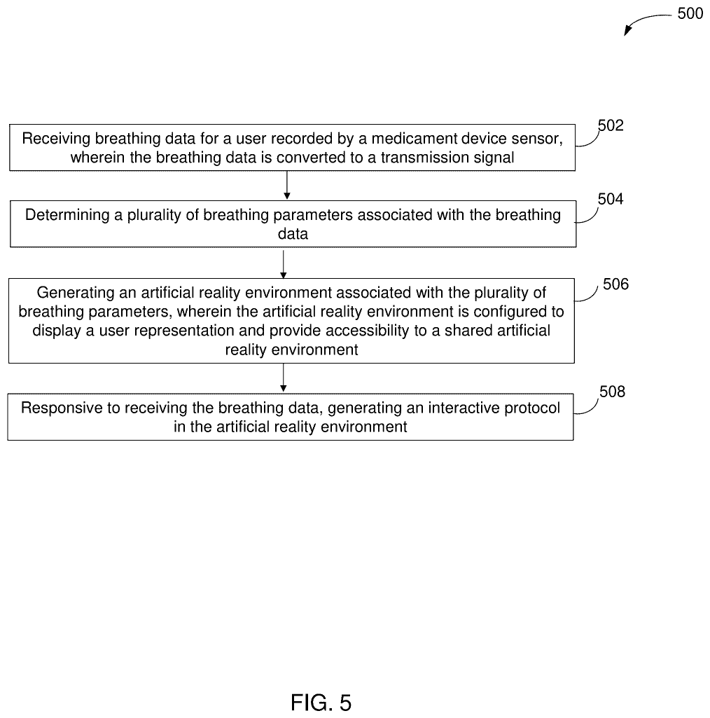

FIG. 5 is an example flow diagram (e.g. the process 500) for implementing a medicament device in conjunction with an artificial reality environment. At step 502, the medicament device can receive breathing data for a user recorded by a medicament device sensor, where in the breathing data is converted to a transmission signal. At step 504, the medicament device can determine a plurality of breathing parameters associated with the breathing data. At step 506, the medicament device in communication with a second processor of a computing device external to the medicament device in conjunction with an artificial reality headset to generate an artificial reality environment associated with the plurality of breathing parameters, wherein the artificial reality environment is configured to display a user representation and provide accessibility to a shared artificial reality environment. At step 508, in to receiving the breathing data, the medicament device can generate an interactive protocol in the artificial reality environment. In an alternative, the second processor can generate the alternative protocol.

The techniques described herein may be implemented as method(s) that are performed by physical computing device(s); as one or more non-transitory computer-readable storage media storing instructions which, when executed by computing device(s), cause performance of the method(s); or as physical computing device(s) that are specially configured with a combination of hardware and software that causes performance of the method(s).

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the terms “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

What is claimed is:

1. An inhaler monitoring system comprising:
a medicament device configured to provide medication to a user wherein the medicament device comprises:
a mouthpiece, wherein the mouthpiece comprise a sensor configured to measure an airflow through the mouthpiece and generate a signal associated with the airflow, wherein the sensor is oriented parallel to the airflow through the mouthpiece,
a processor and memory for storing instructions that, when executed by the processor, cause the processor to:
transmit the signal from the sensor, and
facilitate an artificial reality environment; and
an artificial reality headset configured to permit the user to interact in the artificial reality environment.

2. The inhaler monitoring system of claim 1, wherein the medicament device is a nebulizer.

3. The inhaler monitoring system of claim 1, wherein the sensor measures barometric pressure.

4. The inhaler monitoring system of claim 1, wherein the sensor comprises a microphone.

5. The inhaler monitoring system of claim 1, wherein the artificial reality environment is configured to be shared with at least other users and/or a health care provider.

6. The inhaler monitoring system of claim 1, wherein the instructions to facilitate the artificial reality environment are based on a plurality of breathing measurements.

7. The inhaler monitoring system of claim 1, wherein the instructions to facilitate the artificial reality environment comprise an adherence protocol.

8. A method for using an inhaler monitoring system, the system comprising a medicament device and an artificial reality headset, wherein the method comprises:
receiving breathing data for a user recorded by a medicament device sensor, where in the breathing data is converted to a transmission signal;
determining a plurality of breathing parameters associated with the breathing data;
generating an artificial reality environment associated with the plurality of breathing parameters, wherein the artificial reality environment is configured to display a user representation and provide accessibility to a shared artificial reality environment; and
responsive to receiving the breathing data, generating an interactive protocol in the artificial reality environment.

9. The method of claim 8, wherein generating an interactive protocol comprises at least an adherence protocol involving use of the medicament device.

10. The method of claim 8 further comprising, comparing the breathing parameters to a threshold for each of the plurality of breathing parameters.

11. The method of claim 8 further comprising generating a real-time notification of the breathing data and transmitting the notification to a client device.

12. The method of claim 8, wherein the artificial reality environment is configured to be shared with at least other users or a health care provider.

13. The method of claim 8, wherein determining a plurality of breathing parameters associated with the breathing data comprises a barometric pressure.

14. The method of claim 8 further comprising transmitting the breathing data to a remote computing device.

15. The method of claim 8, wherein artificial reality comprises at least one of: virtual reality, augmented reality, or mixed reality.

16. A non-transitory computer readable storage medium comprising computer program instructions that when executed by a computer processor cause the processor to:
receive breathing data for a user recorded by a medicament device sensor, where in the breathing data is converted to a transmission signal;
determine a plurality of breathing parameters associated with the breathing data;
generate an artificial reality environment associated with the plurality of breathing parameters for the artificial reality, wherein the artificial reality environment is configured to display a user representation and provide accessibility to a shared artificial reality environment; and
responsive to receiving the breathing data, generating an interactive protocol in the artificial reality environment.

17. The non-transitory computer readable storage medium of claim 16, wherein the artificial reality environment is configured to be shared with at least other users and/or a health care provider.

18. The non-transitory computer readable storage medium of claim 16, wherein the plurality of breathing parameters associated with the breathing data comprises a barometric pressure.

19. The non-transitory computer readable storage medium of claim 16, wherein the instructions are further configured to execute comparing the breathing parameters to a threshold for each of the plurality of breathing parameters.

20. The non-transitory computer readable storage medium of claim 16, wherein the instructions are further configured to generate a real-time notification of the breathing data and transmitting the notification to a client device.
